(19) United States
(12) Patent Application Publication
(10) Pub. No.: US 2012/0232172 A1
(43) Pub. Date: Sep. 13, 2012

(54) HYDROCRACKING PROCESS AND PROCESS FOR PRODUCING HYDROCARBON OIL

(76) Inventors: Kazuhiko Tasaka, Tokyo (JP); Yuichi Tanaka, Tokyo (JP); Marie Iwama, Tokyo (JP)

(21) Appl. No.: 13/508,128

(22) PCT Filed: Oct. 26, 2010
(86) PCT No.: PCT/JP2010/068916

§ 371 Date: May 4, 2012

(30) Foreign Application Priority Data
Nov. 9, 2009 (JP) 2009-256123

(51) Int. Cl. C07C 27/00; C10G 67/02

(52) U.S. Cl. 518/700; 208/95

(57) ABSTRACT

A hydrocracking process for a wax fraction that includes a wax fraction hydrocracking step of hydrocracking a wax fraction contained within liquid hydrocarbons synthesized by a Fischer-Tropsch synthesis reaction, thereby obtaining a hydrocracked product, a fractional distillation step of supplying the hydrocracked product to a fractionator in which a bottom cut temperature is set to a constant value, and obtaining at least a middle distillate and a bottom oil from the fractionator, a recycling step of resupplying all of the bottom oil to the wax fraction hydrocracking step, and a hydrocracking control step of controlling the wax fraction hydrocracking step using a flow rate of the bottom oil as an indicator.

### TECHNICAL FIELD

The present invention relates to a hydrocracking process for hydrocracking a wax fraction contained within a synthetic oil produced by a Fischer-Tropsch synthesis reaction, and also relates to a process for producing a hydrocarbon oil.

Priority is claimed on Japanese Patent Application No. 2009-256123, filed Nov. 9, 2009, the content of which is incorporated herein by reference.

### BACKGROUND ART

In recent years, the desire to reduce environmental impact has resulted in growing demands for clean liquid fuels that contain minimal amounts of sulfur and aromatic hydrocarbons and are gentle on the environment. As a result of these demands, processes that employ a Fischer-Tropsch synthesis reaction (hereafter abbreviated as “FT synthesis reaction”), which uses a gas containing carbon monoxide gas and hydrogen gas as a feedstock, have begun to be investigated as potential processes that are capable of producing fuel oil base stocks, and particularly kerosene and gas oil base stocks, that contain minimal sulfur and aromatic hydrocarbons and are rich in aliphatic hydrocarbons (for example, see Patent Document 1).

As a process for producing liquid fuel base stocks using FT synthesis reaction, GTL (Gas To Liquids) process has been known, which produces a synthesis gas containing carbon monoxide gas and hydrogen gas as main components by reforming reaction using a gaseous hydrocarbon such as natural gas as a feedstock, synthesizes a synthetic oil comprising liquid hydrocarbons, and further hydroprocesses and fractionally distills the synthetic oil to obtain hydrocarbon oils used as liquid fuel base stocks.

The synthetic oil (raw oil) obtained by the FT synthesis reaction (hereafter referred to as “FT synthetic oil”) is a mixture containing mainly aliphatic hydrocarbons having a broad carbon number distribution. From this FT synthetic oil can be obtained a naphtha fraction containing a large amount of components having a boiling point lower than approximately 150° C., a middle distillate containing a large amount of components having a boiling point within a range from approximately 150° C. to approximately 360° C., and a wax fraction (hereafter also referred to as the “FT wax fraction”) containing those hydrocarbon components that are heavier than the middle distillate (namely, components having a boiling point that exceeds approximately 360° C.). Of these fractions, the middle distillate is the most useful fraction, being equivalent to a kerosene and gas oil base stock, and it is desirable to achieve a high yield of this middle distillate. Accordingly, in an upgrading step used for obtaining fuel oil base stocks from the FT synthetic oil, the FT wax fraction, which is produced in a reasonably large amount together with the middle distillate during the FT synthesis reaction step, is subjected to hydrocracking to reduce the molecular weight and convert the wax fraction components to components equivalent to the middle distillate, thereby increasing the overall yield of the middle distillate.

In the wax fraction hydrocracking step, if the reaction conditions are severe, causing an increase in the degree of progression of the hydrocracking, then a portion of the wax fraction undergoes excessive cracking, resulting in increased production of a naphtha fraction or gaseous hydrocarbons that are lighter than the targeted middle distillate, meaning the yield of the middle distillate is reduced. Accordingly, the conditions for the hydrocracking reaction are generally selected so as to maximize the proportion of those products within the hydrocracked product that belong to the middle distillate region. Under these types of hydrocracking reaction conditions, a portion of the wax fraction undergoes insufficient cracking, and remains within the cracked product as uncracked wax fraction. This uncracked wax fraction is recovered by fractional distillation from the hydrocracked product obtained in the wax fraction hydrocracking step, and is then resupplied to the wax fraction hydrocracking step.

In the description of the present invention, unless stated otherwise, the expression “hydrocracked product” refers to the entire outflow from the wax fraction hydrocracking step, which includes not only hydrocarbon components having a molecular weight that has fallen below a predetermined level as a result of the hydrocracking, but also the aforementioned uncracked wax fraction.

Specifically, the FT wax fraction that is obtained from fractional distillation of the FT synthetic oil is subjected to hydrocracking in a wax fraction hydrocracking step, and subsequently undergoes gas-liquid separation in a gas-liquid separation step. The thus obtained liquid component (hydrocarbon oil) is fed into a later stage fractionator together with the middle distillate, which has previously been fractionally distilled from the FT synthetic oil and subjected to a separate hydrotreating, and the combined fractions are then subjected to fractional distillation to obtain a middle distillate (kerosene and gas oil fraction). At this time, a heavy component (bottom oil) containing uncracked wax fraction as the main component is recovered from the bottom of the fractionator. All of this bottom oil is recycled, and is resupplied, together with the wax fraction from the FT synthesis reaction step, to the wax fraction hydrocracking step, where it is once again subjected to hydrocracking (for example, see Patent Document 2).

In this manner, by adjusting the degree of progression of the cracking in the wax fraction hydrocracking step, and resupplying the bottom oil from the fractionator to the wax fraction hydrocracking step, so that the bottom oil is converted to components equivalent to the middle distillate, the final yield of the middle distillate can be further increased.

### CITATION LIST

### Patent Document

[Patent Document 1] Japanese Patent Unexamined Publication No. 2004-323626
[Patent Document 2] Japanese Patent Unexamined Publication No. 2007-204506

### SUMMARY OF INVENTION

### Technical Problem

However, conventionally, when a bottom oil is recovered from a fractionator in this manner and then resupplied to the wax fraction hydrocracking step, for reasons of operational simplicity, the fractionator has typically been controlled so that the flow rate of the recovered and resupplied bottom oil remains constant. If this type of fractionator control is employed, then if the properties (mainly the composition distribution) of the hydrocarbon oil supplied to the fractionator fluctuate for some reason, the properties of the bottom oil discharged from the fractionator also fluctuate, and regardless of those fluctuations, a constant volume of the bottom oil is still resupplied to the wax fraction hydrocracking step. As a result, if the properties of the hydrocarbon oil supplied to the fractionator fluctuate once, then a type of vicious cycle described below is established which amplifies the fluctuation, and may finally lead to a situation where the quality of the kerosene and gas oil base stock that represents the product from the fractionator is adversely affected.

In other words, if, for some reason, the composition of the hydrocarbon oil being supplied to the fractionator changes to a composition containing lighter components than normal, then the bottom oil obtained from the fractionator will also become lighter. This lighter bottom oil is then resupplied to the hydrocracking step and subjected to further hydrocracking, causing further lightening of the oil, and as a result, an even lighter hydrocarbon oil is supplied to the fractionator, establishing a vicious cycle. If the hydrocarbon oil supplied to the fractionator is lightened, then the product obtained as a kerosene and gas oil base stock will also become lighter, causing concern over factors such as the kinetic viscosity of the product. In contrast, if, for some reason, the composition of the hydrocarbon oil being supplied to the fractionator changes to a composition containing heavier components than normal, then the bottom oil obtained from the fractionator will also become heavier. If this type of heavier bottom oil is resupplied to the hydrocracking step, then the hydrocracking tends to be insufficient, and as a result, a heavier hydrocarbon oil that has undergone insufficient hydrocracking is supplied to the fractionator, establishing a vicious cycle. If the hydrocarbon oil supplied to the fractionator becomes overly heavy, then there is a possibility that heavy components not normally contained within the kerosene and gas oil fraction may become incorporated within the fraction, causing a deterioration in the low-temperature fluidity properties of the product such as the pour point.

In those cases where a fractionator is controlled so that the flow rate of the bottom oil is maintained at a constant level, if the properties of the hydrocarbon oil being supplied to the fractionator fluctuate once from the standard properties, then the type of vicious cycle described above is established, which amplifies the fluctuation and raises concern about potential adverse effects on the quality of the products.

Examples of potential causes of fluctuations in the properties of the hydrocarbon oil supplied to the fractionator include fluctuations in the wax fraction hydrocracking step such as deterioration of the hydrocracking catalyst used in the wax fraction hydrocracking step, and property fluctuations in the FT synthetic oil caused by fluctuations in the conditions for the FT synthesis reaction step.

Further, sampling the hydrocarbon oil supplied to the fractionator, and then analyzing the sample to enable fluctuations in the composition to be ascertained in “real time” is unrealistic due to the complexity of the sampling operation and the time required for the analysis.

The present invention has been developed in light of the above circumstances, and has an object of providing a hydrocracking process for a wax fraction in which a bottom oil obtained from a fractionator is resupplied to a wax fraction hydrocracking step, wherein even if the properties of the hydrocarbon oil supplied to the fractionator fluctuate from the standard properties, a vicious cycle that amplifies the fluctuation is suppressed, and the properties of the hydrocarbon oil supplied to the fractionator is rapidly stabilized at the standard properties, meaning the quality of the product obtained from the fractionator can be stably maintained. The present invention also provides a process for producing a hydrocarbon oil using said hydrocracking process for a wax fraction.

### Solution to Problem

The inventors of the present invention focused their attention on a hydrocracking process for a wax fraction in which a bottom oil obtained from a fractionator is resupplied to a wax fraction hydrocracking step, and discovered that by controlling the bottom cut temperature of the fractionator at a constant level, instead of controlling the fractionator so that the flow rate of the bottom oil was maintained at a constant level, the properties of the bottom oil were kept constant regardless of any fluctuations in the properties of the hydrocarbon oil supplied to the fractionator. If the properties of the bottom oil are kept constant in this manner, then the properties of the hydrocracked product obtained from the wax fraction hydrocracking step to which the bottom oil is resupplied also become constant.

Further, in those cases where the bottom cut temperature is controlled at a constant level in this manner, if the properties of the hydrocarbon oil supplied to the fractionator fluctuate, then the flow rate of the bottom oil will undergo a corresponding fluctuation. Accordingly, by focusing on the flow rate of the bottom oil, any fluctuations in the properties of the hydrocarbon oil being supplied to the fractionator can be detected promptly without having to analyze the hydrocarbon oil. Consequently, the inventors conceived of a process in which by using this bottom oil flow rate as an indicator, and adjusting the reaction conditions of the wax fraction hydrocracking step accordingly, the degree of progression of the hydrocracking in the wax fraction hydrocracking step was able to be controlled at an appropriate level, and the properties of the hydrocracked product obtained from the wax fraction hydrocracking step were also able to be maintained at a constant level, and they were therefore able to complete the present invention.

In other words, a hydrocracking process for a wax fraction according to the present invention includes:

a wax fraction hydrocracking step of hydrocracking a wax fraction contained within liquid hydrocarbons synthesized by a Fischer-Tropsch synthesis reaction, thereby obtaining a hydrocracked product,

a fractional distillation step of supplying the hydrocracked product to a fractionator in which a bottom cut temperature is set to a constant value, and obtaining at least a middle distillate and a bottom oil from the fractionator,

a recycling step of resupplying all of the bottom oil to the wax fraction hydrocracking step, and

a hydrocracking control step of controlling the wax fraction hydrocracking step using a flow rate of the bottom oil as an indicator.

A process for producing a hydrocarbon oil according to the present invention includes:

a liquid hydrocarbon synthesis step of synthesizing liquid hydrocarbons from a feedstock gas containing carbon monoxide gas and hydrogen gas by a Fischer-Tropsch synthesis reaction,

a wax fraction hydrocracking step of hydrocracking a wax fraction contained within the liquid hydrocarbons synthesized in the liquid hydrocarbon synthesis step, thereby obtaining a hydrocracked product,

a fractional distillation step of supplying the hydrocracked product to a fractionator in which a bottom cut temperature is set to a constant value, and obtaining at least a middle distillate and a bottom oil from the fractionator,

a recycling step of resupplying all of the bottom oil to the wax fraction hydrocracking step, and

a hydrocracking control step of controlling the wax fraction hydrocracking step using a flow rate of the bottom oil as an indicator.

In the hydrocracking control step, a relationship between the flow rate of the bottom oil and a reaction temperature of the wax fraction hydrocracking step may be ascertained in advance, and the reaction temperature may be then set in accordance with the flow rate of the bottom oil based on this relationship.

Furthermore, in the hydrocracking control step, a flow rate of the wax fraction may be adjusted in accordance with the flow rate of the resupplied bottom oil, so that a combined flow rate of the wax fraction that is supplied to the wax fraction hydrocracking step and the bottom oil that is resupplied to the wax fraction hydrocracking step remains constant.

### Advantageous Effects of Invention

According to the present invention, in a hydrocracking process for a wax fraction in which a bottom oil obtained from a fractionator is resupplied to a wax fraction hydrocracking step, even if the properties of the hydrocarbon oil supplied to the fractionator fluctuate from the standard properties, it is possible that a vicious cycle which amplifies the fluctuation is suppressed, and that the properties of the hydrocarbon oil supplied to the fractionator is rapidly stabilized at the standard properties. As a result, there are provided a hydrocracking process for a wax fraction and a process for producing a hydrocarbon oil, wherein the quality of the middle distillate product obtained from the fractionator can be stably maintained.

### BRIEF DESCRIPTION OF THE DRAWINGS

FIG. 1 is a schematic diagram illustrating a liquid fuel synthesizing system performing GTL process.

FIG. 2 is a diagram illustrating specifics of a upgrading unit producing liquid fuel base stocks which is a portion of FIG. 1.

FIG. 3 is a graph illustrating the relationship between the flow rate of bottom oil, and the reaction temperature (actual measured value) of the wax fraction hydrocracking step that gives such a bottom oil flow rate.

### DESCRIPTION OF EMBODIMENTS

A more detailed description of the present invention is presented below.

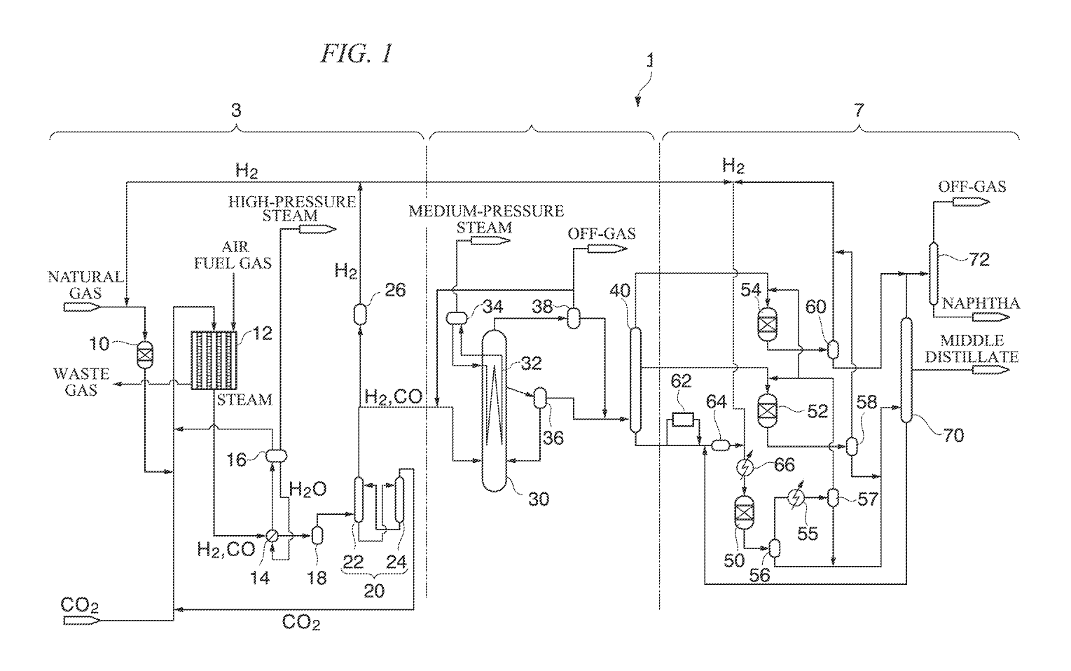

FIG. 1 illustrates a liquid fuel synthesizing system 1 that carries out a GTL process for converting a natural gas as a hydrocarbon feedstock to liquid fuel base stocks. This liquid fuel synthesizing system 1 is composed of a synthesis gas production unit 3, an FT synthesis unit 5, and an upgrading unit 7.

The synthesis gas production unit 3 reforms a natural gas that functions as a hydrocarbon feedstock to produce a synthesis gas containing carbon monoxide gas and hydrogen gas.

The FT synthesis unit 5 synthesizes liquid hydrocarbons from the produced synthesis gas via a FT synthesis reaction.

The upgrading unit 7 hydroprocesses and fractionally distills the liquid hydrocarbons synthesized by the FT synthesis reaction to produce hydrocarbon oils used for base stocks for liquid fuels (such as naphtha, kerosene, gas oil and wax). Components of each of these units are described below.

The synthesis gas production unit 3 is composed mainly of a desulfurization reactor 10, a reformer 12, a waste heat boiler 14, gas-liquid separators 16 and 18, a CO₂ removal unit 20, and a hydrogen separator 26.

The desulfurization reactor 10 is composed of a hydrodesulfurizer and the like, and removes sulfur components from the natural gas that functions as the feedstock.

The reformer 12 reforms the natural gas supplied from the desulfurization reactor 10 to produce a synthesis gas containing carbon monoxide gas (CO) and hydrogen gas (H₂) as main components.

The waste heat boiler 14 recovers waste heat from the synthesis gas produced in the reformer 12 to generate a high-pressure steam.

The gas-liquid separator 16 separates the water that has been heated by heat exchange with the synthesis gas in the waste heat boiler 14 into a gas (high-pressure steam) and a liquid.

The gas-liquid separator 18 removes a condensed component from the synthesis gas that has been cooled in the waste heat boiler 14, and supplies a gas component to the CO₂ removal unit 20.

The CO₂ removal unit 20 has an absorption tower 22 that uses an absorbent to remove carbon dioxide gas from the synthesis gas supplied from the gas-liquid separator 18, and a regeneration tower 24 that releases the carbon dioxide gas absorbed by the absorbent, thereby regenerating the absorbent.

The hydrogen separator 26 separates a portion of the hydrogen gas contained within the synthesis gas from the synthesis gas, from which the carbon dioxide gas has already been separated by the CO₂ removal unit 20.

The FT synthesis unit 5 includes, for example, a bubble column reactor (a bubble column hydrocarbon synthesis reactor) 30, a gas-liquid separator 34, a separator 36, and a first fractionator 40.

The bubble column reactor 30 is an example of a reactor that synthesizes liquid hydrocarbons from a synthesis gas, and functions as an FT synthesis reactor that synthesizes liquid hydrocarbons from the synthesis gas by the FT synthesis reaction. This bubble column reactor 30 may be composed, for example, from a bubble column slurry bed type reactor in which a catalyst slurry prepared by suspending solid catalyst particles within liquid hydrocarbons (the FT synthesis reaction product) is contained in a column type vessel. This bubble column reactor 30 synthesizes liquid hydrocarbons by reacting the carbon monoxide gas and hydrogen gas contained within the synthesis gas produced in the aforementioned synthesis gas production unit 3.

The gas-liquid separator 34 separates the water that has been heated by passage through a heat transfer tube 32 provided inside the bubble column reactor 30 into a steam (medium-pressure steam) and a liquid.

The separator 36 separates the catalyst slurry contained in the bubble column reactor 30 into the catalyst particles and the liquid hydrocarbons.

The first fractionator 40 fractionally distills the liquid hydrocarbons, which have been supplied from the bubble column reactor 30 via the separator 36 and the gas-liquid separator 38, into respective fractions.

The upgrading unit 7 includes, for example, a wax fraction hydrocracking reactor 50, a middle distillate hydrotreating reactor 52, a naphtha fraction hydrotreating reactor 54, gas-liquid separators 56, 57, 58 and 60, a second fractionator 70, and a naphtha stabilizer 72.

The wax fraction hydrocracking reactor 50 is connected to the bottom of the first fractionator 40, with the first gas-liquid separator 56 and second gas-liquid separator provided in a multiple stage downstream from the wax fraction hydrocracking reactor 50.

The middle distillate hydrotreating reactor 52 is connected to a middle section of the first fractionator 40, with the gas-liquid separator 58 provided downstream from the middle distillate hydrotreating reactor 52.

The naphtha fraction hydrotreating reactor 54 is connected to the top of the first fractionator 40, with the gas-liquid separator 60 provided downstream from the naphtha fraction hydrotreating reactor 54.

The second fractionator 70 fractionally distills the mixture of the hydrocarbon oils supplied from the first gas-liquid separators 56, the second gas-liquid separator 57 and the gas-liquid separator 58 in accordance with the boiling points.

The naphtha stabilizer 72 further fractionally distills the hydrocarbon oil within the naphtha fraction supplied from the gas-liquid separator 60 and the second fractionator 70, and the resulting light component is discharged as an off-gas, while the heavy component is separated and recovered as a naphtha product.

Next is a description of a process for producing hydrocarabon oils used for base stocks for liquid fuels from a natural gas (GTL process) using the liquid fuel synthesizing system 1 having the configuration described above.

A natural gas (the main component of which is CH₄) is supplied as a hydrocarbon feedstock to the liquid fuel synthesizing system 1 from an external natural gas supply source (not shown in the drawing), such as a natural gas field or a natural gas plant. The above synthesis gas production unit 3 reforms the natural gas to produce a synthesis gas (a mixed gas containing carbon monoxide gas and hydrogen gas as main components).

Specifically, first, the natural gas described above is introduced into the desulfurization reactor 10 together with the hydrogen gas separated by the hydrogen separator 26. In the desulfurization reactor 10, sulfur components contained in the natural gas are converted into a hydrogen sulfide by the introduced hydrogen gas under the action of a conventional hydrodesulfurization catalyst, and the thus generated hydrogen sulfide is adsorbed by an absorber such as ZnO. As a result, the sulfur components are removed from the natural gas.

The desulfurized natural gas is supplied to the reformer 12 after mixing with carbon dioxide gas (CO₂) supplied from a carbon dioxide supply source (not shown in the drawing) and the steam generated in the waste heat boiler 14. In the reformer 12, the natural gas is reformed by the carbon dioxide gas and the steam via a steam-carbon dioxide reforming process, thereby producing a high-temperature synthesis gas containing carbon monoxide gas and hydrogen gas as main components.

The high-temperature synthesis gas (for example, 900° C., 2.0 MPaG) produced in the reformer 12 in this manner is supplied to the waste heat boiler 14, and is cooled (for example, to 400° C.) by heat exchange with the water circulating through the waste heat boiler 14, thereby recovering the waste heat from the synthesis gas.

The synthesis gas that has been cooled within the waste heat boiler 14 is supplied to either the absorption tower 22 of the CO₂ removal unit 20 or the bubble column reactor 30, after a condensed liquid fraction has been separated and removed from the synthesis gas in the gas-liquid separator 18. In the absorption tower 22, carbon dioxide gas contained in the synthesis gas is absorbed by an absorbent, and this carbon dioxide gas is then released from the absorbent in the regeneration tower 24. The released carbon dioxide gas is fed from the regeneration tower 24 into the reformer 12, and is reused for the above reforming reaction.

The synthesis gas produced in the synthesis gas production unit 3 in this manner is supplied to the bubble column reactor 30 of the aforementioned FT synthesis unit 5. At this time, the composition ratio of the synthesis gas supplied to the bubble column reactor 30 is adjusted to a composition ratio suitable for the FT synthesis reaction (for example, H₂:CO=2:1 (molar ratio)).

In the hydrogen separator 26, the hydrogen gas contained in the synthesis gas is separated by adsorption and desorption utilizing a pressure difference (hydrogen PSA). The separated hydrogen gas is supplied continuously from a gas holder or the like (not shown in the drawing) via a compressor (not shown in the drawing) to the various hydrogen-utilizing reactors (for example, the desulfurization reactor 10, the wax fraction hydrocracking reactor 50, the middle distillate hydrotreating reactor 52, and the naphtha fraction hydrotreating reactor 54) within the liquid fuel synthesizing system 1 that perform predetermined reactions by utilizing hydrogen gas.

Next, the FT synthesis unit 5 synthesizes liquid hydrocarbons by the FT synthesis reaction from the synthesis gas produced in the above synthesis gas production unit 3.

Specifically, the synthesis gas produced in the synthesis gas production unit 3 is introduced into the bottom of the bubble column reactor 30, and rises up through the catalyst slurry contained within the bubble column reactor 30. During this time within the bubble column reactor 30, the carbon monoxide gas and hydrogen gas contained within the synthesis gas react with each other by the aforementioned FT synthesis reaction, and liquid hydrocarbons are produced.

The liquid hydrocarbons synthesized in the bubble column reactor 30 are introduced into the separator 36 with catalyst particles as a catalyst slurry.

In the separator 36, the introduced catalyst slurry is separated into a solid component composed of the catalyst particles and the like and a liquid component containing the liquid hydrocarbons. A portion of the separated solid component composed of the catalyst particles and the like is returned to the bubble column reactor 30, and the liquid component is supplied to the first fractionator 40.

A gaseous by-product, which contains hydrocarbon compounds generated which is gaseous under the conditions in the bubble column reactor 30 and unreacted synthesis gas, is discharged from the top of the bubble column reactor 30 and supplied to the gas-liquid separator 38. In the gas-liquid separator 38, this gaseous by-product is cooled, and condensed light liquid hydrocarbons are separated and introduced into the first fractionator 40. The gas component separated by the gas-liquid separator 38 contains the unreacted synthesis gases (CO and H₂) and hydrocarbon gases with a carbon number of 4 or less as main components, and a portion of this gas component is reintroduced into the bottom of the bubble column reactor 30, so that the unreacted synthesis gas therein is reused for the FT synthesis reaction. Further, the gas component that is not reintroduced into the bubble column reactor 30 is discharged as an off-gas, which may be used as a fuel gas, treated for the recovery of fuels equivalent to LPG (Liquefied Petroleum Gas), or reused as a feedstock for the reformer 12 of the synthesis gas production unit.

There are no particular limitations on the liquid hydrocarbons obtained in the FT synthesis unit 5, that are to be used as a feedstock for the production of hydrocarbon oils used as liquid fuel base stocks within the upgrading unit 7. However, in terms of maximizing the yield of the middle distillate, the liquid hydrocarbons preferably contain at least 80 mass % of hydrocarbons with a boiling point of approximately 150° C. or higher based on the total mass of the liquid hydrocarbons obtained by the FT synthesis reaction.

Subsequently, in the first fractionator 40, the liquid hydrocarbons supplied from the bubble column reactor 30 via the separator 36 and the gas-liquid separator 38 in the manner described above are fractionally distilled into a naphtha fraction (with a boiling point that is lower than approximately 150° C.), a middle distillate equivalent to a kerosene and gas oil fraction (with a boiling point of approximately 150 to 360° C.), and a wax fraction (with a boiling point that exceeds approximately 360° C.).

Additionally, this description describes a preferred embodiment in which two cut points (namely, at approximately 150° C. and approximately 360° C.) are set in the first fractionator 40, thereby separating the liquid hydrocarbons into three fractions. However, for example, a single cut point may also be set, in which case the fraction that distills at a temperature below the cut point is discharged from the middle section of the first fractionator 40 as the middle distillate and the fraction with a boiling point exceeding the cut point is discharged from the bottom of the first fractionator 40 as the wax fraction.

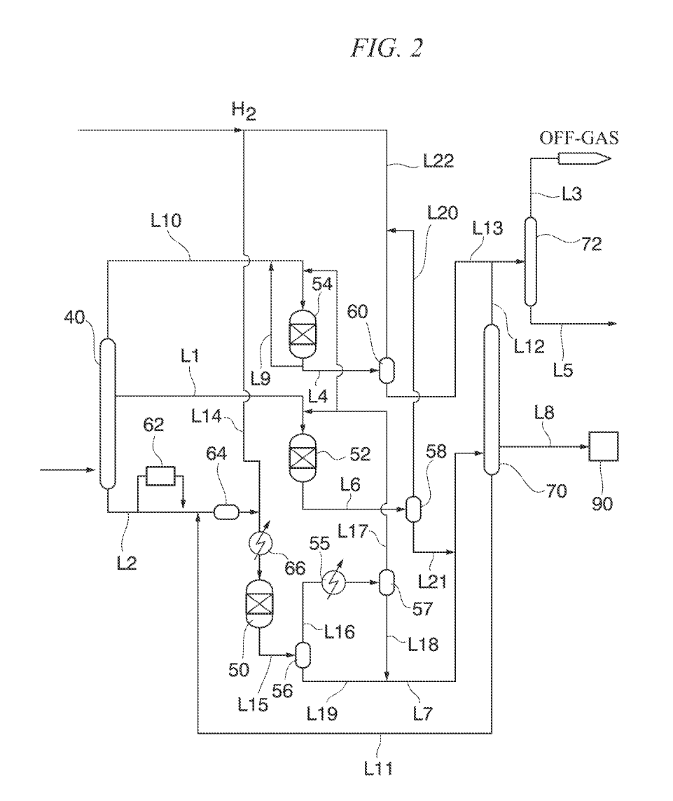

A hydrocarbon oil producing process in the upgrading unit 7 is described below with reference to FIG. 2, which illustrates details of the upgrading unit 7.

The upgrading unit 7 produces hydrocarbon oils used as base stocks for liquid fuels (naphtha, kerosene, gas oil, wax, and etc.) by hydroprocessing and further fractionally distilling each of the liquid hydrocarbons synthesized in the FT synthesis unit 5 and fractionally distilled in the first fractionator.

The liquid hydrocarbon compounds of the naphtha fraction (mainly hydrocarbons of C₅ to C₁₀) discharged from the top of the first fractionator 40 are brought into the naphtha fraction hydrotreating reactor 54 through a line L10. The liquid hydrocarbon compounds of the middle distillate (mainly hydrocarbons of C₁₁ to C₂₀) discharged from the middle section of the first fractionator 40 are brought into the middle distillate hydrotreating reactor 52 through a line L1. The liquid hydrocarbons of the wax fraction (mainly hydrocarbons of C₂₁ or more) discharged from the bottom of the first fractionator 40 are brought into the wax fraction hydrocracking reactor 50 through a line L2.

In the naphtha fraction hydrotreating reactor 54, the liquid hydrocarbons of the naphtha fraction having a low carbon number (of approximately C₁₀ or less) that have been discharged from the top of the first fractionator 40 are hydrotreated using hydrogen gas supplied from the hydrogen separator 26 via the wax fraction hydrocracking reactor 50. During the hydrotreating, olefins and oxygen-containing compounds such as alcohols, that are produced as by-products in the FT synthesis reaction and contained in the liquid hydrocarbons of the naphtha fraction, are respectively hydrogenated and hydrodeoxygenated to be converted into paraffinic hydrocarbons. The product containing the hydrotreated hydrocarbon oil is separated into a gas component and a liquid component in the gas-liquid separator 60. The separated liquid component is brought into the naphtha stabilizer 72 through a line L13, and the separated gas component (containing hydrogen gas) is supplied to the wax fraction hydrocracking reactor through lines L22 and L14 and the hydrogen gas therein is reused.

Additionally, a portion of the hydrotreated naphtha fraction discharged from the naphtha fraction hydrotreating reactor 54 is passed through a line L9 and recycled to the line L10 upstream from the naphtha fraction hydrotreating reactor 54. The hydrotreating of the naphtha fraction is a highly exothermic reaction, and if only the untreated naphtha fraction is subjected to the hydrotreating, then there is possibility that the temperature of the naphtha fraction in the naphtha fraction hydrotreating reactor 54 may rise excessively. Accordingly, by recycling a portion of the hydrotreated naphtha fraction, the untreated naphtha fraction is diluted, thereby preventing any excessive temperature rising.

In the middle distillate hydrotreating reactor 52, the liquid hydrocarbons of the middle distillate having a mid-range carbon number (of approximately C₁₁ to C₂₀) that have been discharged from the middle section of the first fractionator 40 are hydrotreated using hydrogen gas supplied from the hydrogen separator 26 via the wax fraction hydrocracking reactor 50. During this hydrotreating, the olefins and oxygen-containing compounds such as alcohols are respectively hydrogenated and hydrodeoxygenated to be converted into paraffinic hydrocarbons, and at least a portion of normal paraffins are hydroisomerized to form isoparaffins. According to the hydroisomerization of the normal paraffins into isoparaffins, low-temperature fluidity of the hydrotreated hydrocarbons of middle distillate as a fuel base stock is improved.

The product containing the hydrotreated hydrocarbon oil is separated into a gas component and a liquid component in the gas-liquid separator 58. The separated liquid component is brought into the second fractionator 70, and the separated gas component (containing hydrogen gas) is supplied to the wax fraction hydrocracking reactor through lines L20, L22 and L14 and the hydrogen gas therein is reused.

In the wax fraction hydrocracking reactor 50, the liquid hydrocarbons of the wax fraction (hydrocarbons of approximately C₂₁ or more) discharged from the bottom of the first fractionator 40 are hydrocracked by using the hydrogen gas supplied from the above hydrogen separator 26, the naphtha fraction hydrotreating reactor 54, and the middle distillate hydrotreating reactor 52. During the hydrocracking, the carbon number of the wax fraction is reduced to approximately 20 or less and the wax fraction is converted into a fraction equivalent to middle distillate. The olefins and oxygen-containing compounds such as alcohols contained within the liquid hydrocarbons of wax fraction are converted into paraffinic hydrocarbons. Furthermore, at the same time, the production of isoparaffins by hydroisomerization of normal paraffins also proceeds, which contributes to an improvement in the low-temperature fluidity of the product oil for use as a fuel oil base stock.

On the other hand, a portion of the wax fraction undergoes excessive hydrocracking, and is converted into hydrocarbons equivalent to the naphtha fraction having an even lower boiling point than the boiling point range of hydrocarbons equivalent to the targeted middle distillate. Furthermore, a portion of the wax fraction undergoes even more hydrocracking, and is converted to gaseous hydrocarbons with a carbon number of 4 or less, such as butanes, propane, ethane and methane.

The hydrocracking product of the wax fraction discharged from the wax fraction hydrocracking reactor 50 is separated into a gas component and liquid components in a stepwise manner by the multiple stages of the first gas-liquid separators 56 and second gas-liquid separators 57. The separated liquid components (hydrocarbon oils) are brought into the second fractionator 70 from the first gas-liquid separator 56 and second gas-liquid separator 57 respectively, whereas the separated gas component (including hydrogen gas) is supplied to the middle distillate hydrotreating reactor 52 and the naphtha fraction hydrotreating reactor 54 from the second gas-liquid separator 57 through a line L17 and the hydrogen gas therein is reused.

The second fractionator 70 is positioned downstream from the middle distillate hydrotreating reactor 52. Moreover, a middle distillate tank 90 is provided that stores the middle distillate that has been fractionally distilled in the second fractionator 70. The outflow oil from the middle distillate hydrotreating reactor 52 from which the gas component (containing hydrogen gas) has been separated by the gas-liquid separator 58 is supplied to the second fractionator 70 through a line L21. The outflow oil (hydrocracked product) from the wax fraction hydrocracking reactor 50 from which the gas component (containing hydrogen gas) has been separated by the multiple stages of the first gas-liquid separators 56 and second gas-liquid separators 57 is supplied to the second fractionator 70 through a line L19 or line L18 and line L7. The outflow oil from the middle distillate hydrotreating reactor 52 and the outflow oil (hydrocracked product) from the wax fraction hydrocracking reactor 50 that are supplied to the second fractionator 70 may be mixed by either in-line blending or tank blending, and there are no particular limitations on the mixing method employed.

Subsequently, in the second fractionator 70, the mixture of the hydrocarbon oils supplied from the wax fraction hydrocracking reactor 50 and the middle distillate hydrotreating reactor 52 respectively in the manner described above is fractionally distilled into hydrocarbon compounds of C₁₀ or less (with boiling points lower than approximately 150° C.), a middle distillate (with a boiling point of approximately 150 to 360° C.), and an uncracked wax fraction (with a boiling point exceeding approximately 360° C.) which has not been sufficiently hydrocracked in the wax fraction hydrocracking reactor 50. The uncracked wax fraction is mainly obtained from the bottom of the second fractionator 70, and is recycled to a position upstream of the wax fraction hydrocracking reactor 50. The middle distillate is discharged from the middle section of the second fractionator 70. Meanwhile, hydrocarbons of C₁₀ or less are discharged from the top of the second fractionator 70 and supplied to the naphtha stabilizer 72 through lines L12 and L13.

Moreover, in the naphtha stabilizer 72, the hydrocarbons of C₁₀ or less supplied from the naphtha fraction hydrotreating reactor 54 and the second fractionator 70 are fractionally distilled, and naphtha (C₅ to C₁₀) is obtained as a product. Accordingly, high-purity naphtha is discharged from the bottom of the naphtha stabilizer 72. Meanwhile, an off-gas containing hydrocarbons with a carbon number no higher than 4 as main components, namely compounds other than the targeted product, is discharged from the top of the naphtha stabilizer 72. This off-gas may be used as a fuel gas, or treated for the recovery of fuels equivalent to LPG.

In this example, the middle distillate is obtained as a single fraction from the second fractionator 70, and this middle distillate passes through a line L8 and is stored in the middle distillate tank 90. However, the middle distillate may be fractionally distilled into an appropriate plurality of fractions, for example, two fractions such as a kerosene fraction (with a boiling point of approximately 150 to 250° C.) and a gas oil fraction (with a boiling point of approximately 250 to 360° C.), with these fractions then fed into separate tanks for storage.

The bottom oil from the second fractionator 70 is composed mainly of the uncracked wax fraction, namely the wax fraction that has not undergone sufficient hydrocracking during the wax fraction hydrocracking step. This bottom oil is recycled through a line L11 to the line L2 that is upstream from the wax fraction hydrocracking reactor 50, and is once again supplied to the wax fraction hydrocracking reactor 50 and subjected to hydrocracking. This process improves the middle distillate yield.

A hydrocracking process for a the wax fraction is described below with reference to FIG. 2, which illustrates details of the periphery around the wax fraction hydrocracking reactor 50.

In this example, the wax fraction hydrocracking reactor 50 includes a fixed-bed flow reactor, and this reactor is filled with a type of hydrocracking catalyst described below in detail. The FT wax fraction is supplied via the line L2, while hydrogen gas is supplied via a line L14 that connects to the line L2, and these two components are mixed together and then supplied to the wax fraction hydrocracking reactor 50, where the wax fraction undergoes hydrocracking

Further, a multi-stage gas-liquid separator that is described below in detail is provided downstream from the wax fraction hydrocracking reactor 50.

Detailed descriptions of each of the steps in the hydrocracking process for the wax fraction are presented below.

### (Wax Fraction Hydrocracking Step)

As illustrated in FIG. 2, in the wax fraction hydrocracking step, the wax fraction from the FT synthesis reaction step, either supplied from the bottom of the first fractionator, or in some cases supplied via an intermediate tank 62, is subjected to hydrocracking in the wax fraction hydrocracking reactor 50, thus producing a hydrocracked product. At this time, the bottom oil recovered from the bottom of the second fractionator 70 is recycled through the line L11 to the line L2 that is upstream from the wax fraction hydrocracking reactor 50, is subsequently mixed, in a mixing tank 64, with the wax fraction supplied from the first fractionator 40 via the line L2, and is then resupplied to the wax fraction hydrocracking reactor 50 where the bottom oil is once again subjected to hydrocracking. This enables the middle distillate yield to be improved.

Examples of the hydrocracking catalyst used in the wax fraction hydrocracking step include catalysts comprising a metal belonging to one of groups 8 to 10 of the periodic table as an active metal loaded on a support containing a solid acid. The term “periodic table” refers to the long period type periodic table of elements prescribed by IUPAC (the International Union of Pure and Applied Chemistry).

Specific examples of the support include supports containing one or more solid acids selected from among crystalline zeolites such as ultra-stable Y-type (USY) zeolite, Y-type zeolite, mordenite and β-zeolite, and refractory amorphous composite metal oxides such as silica-alumina, silica-zirconia and alumina-boria. The support preferably contains USY zeolite and one or more refractory amorphous composite metal oxides selected from among silica-alumina, alumina-boria and silica-zirconia, and most preferably contains USY zeolite together with alumina-boria and/or silica-alumina.

USY zeolite is prepared by ultra stabilizing a Y-type zeolite via a hydrothermal treatment and/or an acid treatment, and in addition to the micropore structure with a pore size of 2 nm or less inherent to Y-zeolite, USY zeolite also includes new pores having a pore size within a range from 2 to 10 nm. The average particle size of the USY zeolite is not particularly limited, but is preferably not more than 1.0 μm, and more preferably 0.5 μm or less. Further, in the USY zeolite, the silica/alumina molar ratio (the molar ratio of silica relative to alumina) is preferably within a range from 10 to 200, more preferably from 15 to 100, and still more preferably from 20 to 60.

Furthermore, the support preferably contains 0.1 to 80 mass % of the crystalline zeolite and 0.1 to 60 mass % of the refractory amorphous composite metal oxide.

The support can be produced by molding a support composition containing the solid acid described above and a binder, and then calcining the composition. The blend proportion of the solid acid relative to the total mass of the support is preferably within a range from 1 to 70 mass %, and more preferably from 2 to 60 mass %. Furthermore, in those cases where the support includes USY zeolite, the blend proportion of the USY zeolite relative to the total mass of the support is preferably within a range from 0.1 to 10 mass %, and more preferably from 0.5 to 5 mass %. Moreover, in those cases where the support includes USY zeolite and alumina-boria, the blend ratio between the USY zeolite and the alumina-boria (USY zeolite/alumina-boria) is preferably a mass ratio within a range from 0.03 to 1. Further, in those cases where the support includes USY zeolite and silica-alumina, the blend ratio between the USY zeolite and the silica-alumina (USY zeolite/silica-alumina) is preferably a mass ratio within a range from 0.03 to 1.

There are no particular limitations on the binder, although alumina, silica, titania or magnesia is preferred, and alumina is particularly desirable. The blend amount of the binder relative to the total mass of the support is preferably within a range from 20 to 98 mass %, and more preferably from 30 to 96 mass %.

The calcination temperature for the support composition described above is preferably within a range from 400 to 550° C., more preferably from 470 to 530° C., and still more preferably from 490 to 530° C.

Specific examples of the metal belonging to one of groups 8 to 10 of the periodic table include cobalt, nickel, rhodium, palladium, iridium and platinum. Of these, the use of either one metal or a combination of two or more metals selected from among nickel, palladium and platinum is preferred. These metals can be loaded on the aforementioned support using typical methods such as impregnation or ion exchange. Although there are no particular limitations on the amount of metal supported on the support, the total mass of the metal relative to the mass of the support is preferably within a range from 0.1 to 3.0 mass %.

The hydrogen partial pressure in the wax fraction hydrocracking step is typically within a range from 0.5 to 12 MPa, and is preferably from 1.0 to 5.0 MPa.

The liquid hourly space velocity (LHSV) is typically within a range from 0.1 to 10.0 h⁻¹, and is preferably from 0.3 to 3.5 h⁻¹. The ratio between the hydrogen gas and the wax fraction (hydrogen gas/oil ratio) is not particularly limited, but is typically within a range from 50 to 1,000 NL/L, and is preferably from 70 to 800 NL/L.

In this description, the LHSV (liquid hourly space velocity) describes the combined volumetric flow rate of the wax fraction and the resupplied bottom oil from the second fractionator 70 under standard conditions (25° C., 101,325 Pa) per unit volume of the layer of the catalyst (the catalyst layer) charged into the fixed-bed flow reactor, wherein the units “h⁻¹” represent the inverse of “hour”. Further, the units “NL” for the hydrogen gas volume within the hydrogen gas/oil ratio represent the hydrogen gas volume (L) under standard conditions (0° C., 101,325 Pa).

The reaction temperature for the wax fraction hydrocracking step (namely, the catalyst weighted average bed temperature) is typically within a range from 180 to 400° C., and is preferably from 200 to 370° C., more preferably from 250 to 350° C., and still more preferably from 280 to 350° C. If the reaction temperature exceeds 400° C., then the hydrocracking tends to proceed excessively, resulting in a reduction in the yield of the targeted middle distillate. Further, the hydrocracked product may become discolored, placing limits on its potential use as a base stock for fuels. In contrast, if the reaction temperature is lower than 180° C., then the hydrocracking of the wax fraction does not progress sufficiently, and the yield of the middle distillate tends to decrease. Further, the removal of oxygen-containing compounds such as alcohols contained within the wax fraction tends to be inadequate.

The reaction temperature is controlled by adjusting the temperature setting at the outlet of a heat exchanger 66 provided within the line L2.

In this type of wax fraction hydrocracking step, the wax fraction hydrocracking reactor 50 is preferably operated so that the content of a specific hydrocarbon component within the hydrocracked product, namely that hydrocarbon component having a boiling point of not lower than 25° C. and not higher than 360° C., is preferably within a range from 20 to 90 mass %, more preferably from 30 to 80 mass % and still more preferably from 45 to 70 mass %, based on the total mass of the hydrocracked product having a boiling point of 25° C. or higher. Provided the content of this specific hydrocarbon component satisfies the range mentioned above, the degree of progression of the hydrocracking is at an appropriate level, meaning the yield of the middle distillate can be increased.

### (Gas-Liquid Separation Step)

In this example, the hydrocracked product from the wax fraction hydrocracking step is introduced into a multi-stage gas-liquid separator composed of a first gas-liquid separator 56 and a second gas-liquid separator 57. A heat exchanger (not shown in the drawings) for cooling the hydrocracked product is preferably installed within a line L15 connected to the outlet of the wax fraction hydrocracking reactor 50. Following cooling by this heat exchanger, the hydrocracked product is separated into a gas component and a liquid component by the first gas-liquid separator 56. The temperature inside the first gas-liquid separator 56 is preferably approximately 210 to 260° C. In other words, the liquid component separated within the first gas-liquid separator 56 is a heavy oil component composed of hydrocarbons that exist in a liquid state at the above temperature, and includes a large amount of the uncracked wax fraction. This heavy oil component passes out the bottom of the first gas-liquid separator 56, through the line L19 and the line L7, and is supplied to the second fractionator 70.

Meanwhile, the gas component separated within the first gas-liquid separator 56 passes from the top of the first gas-liquid separator 56, through a line L16, to a heat exchanger (cooling device) 55, where it is cooled and at least partially liquefied. The outflow from the heat exchanger 55 is supplied to the second gas-liquid separator 57. As a result of the cooling by the heat exchanger 55, the temperature at the inlet to the second gas-liquid separator 57 is approximately 90 to 100° C.

In the second gas-liquid separator 57, the gas component and the liquid component that has been condensed by the cooling in the heat exchanger 55 are separated. The separated gas component is discharged from the top of the second gas-liquid separator 57 through the line L17. A heat exchanger (not shown in the drawings) is preferably provided within the line L17 to cool the gas component to approximately 40° C. This liquefies a portion of the light hydrocarbons within the gas component, which is then returned to the second gas-liquid separator 57. The remaining gas component is composed mainly of hydrogen gas containing gaseous hydrocarbons, and this gas component is supplied to the middle distillate hydrotreating reactor 52 and the naphtha fraction hydrotreating reactor 54, and reused as hydrogen gas for the hydroprocessing.

Meanwhile, the liquid component is discharged from the line L18 connected to the bottom of the second gas-liquid separator 57. This liquid component is a light oil component composed of lighter hydrocarbons that condense within the second gas-liquid separator 57 at a lower temperature than that within the first gas-liquid separator 56. This light oil component is supplied through the line L7, together with the heavy oil component from the first gas-liquid separator 56, to the second fractionator 70.

By providing the multi-stage gas-liquid separator in this manner, and employing the method described above wherein cooling is performed in a stepwise manner, it is possible to prevent problems such as clogging of the apparatus or the like, which can be caused when the components having a high freezing point (particularly the uncracked wax fraction) within the hydrocracked product from the wax fraction hydrocracking step are solidified by rapid cooling.

### (Fractional Distillation Step)

Subsequently, the liquid component that has been separated from the hydrocracked product of the wax fraction hydrocracking step in the manner described above in the gas-liquid separation step is supplied to the second fractionator 70 via the line L7, and subjected to fractional distillation. A middle distillate (kerosene and gas oil fraction) is discharged through the line L8 connected to the middle section of the second fractionator 70, whereas heavy hydrocarbons containing mainly the residual uncracked wax fraction retained within the hydrocracked product is recovered from the bottom of the fractionator as a bottom oil.

In the fractional distillation step, the second fractionator 70 is operated such that the bottom cut temperature is controlled at a constant value. Here, the “bottom cut temperature” is an indicator of the boundary between the boiling points of the middle distillate and the bottom oil, and for example, may be set as the 10% distillation point, the initial boiling point, or the 5% distillation point in the distillation characteristics of the bottom oil. Furthermore, it may also be set as the 90% distillation point, the 95% distillation point, or the end point for the middle distillate obtained via the line L8. For example, by maintaining the discharge tray temperature of the middle distillate discharged through the line L8 at one of the above temperatures, the bottom cut temperature can be controlled at a constant value.

By controlling the bottom cut temperature at a constant value in this manner, even if, for some reason, the properties of the liquid component (hydrocarbon oil) supplied to the second fractionator 70 from the gas-liquid separation step fluctuate, the properties (composition) of the bottom oil discharged from the second fractionator 70 remain substantially stable. On the other hand, as the properties of the hydrocarbon oil supplied to the second fractionator 70 fluctuate, there is a corresponding fluctuation in the flow rate of the bottom oil discharged from the second fractionator 70.

The bottom cut temperature selected varies depending on the degree of fluctuation in the properties of the hydrocarbon oil supplied to the second fractionator 70, but is typically adjusted to a constant value within a range from 330 to 380° C.

### (Recycling Step)

Subsequently, in the recycling step, all of the bottom oil obtained in the fractional distillation step is resupplied to the wax fraction hydrocracking step. The bottom oil contains the residual uncracked wax fraction that is retained within the hydrocracked product from the wax fraction hydrocracking step, and therefore by resupplying the bottom oil to the wax fraction hydrocracking step in this manner, further hydrocracking of the uncracked wax fraction is able to proceed, enabling the final yield of the middle distillate to be increased.

(Hydrocracking control step)

In the hydrocracking control step, the flow rate of the bottom oil that has been recovered in the fractional distillation step and resupplied to the wax fraction hydrocracking step in the recycling step is used as an indicator to adjust the reaction conditions (such as the reaction temperature) of the wax fraction hydrocracking step, thereby controlling the wax fraction hydrocracking step.

As the reaction temperature of the wax fraction hydrocracking step is raised, the hydrocracking progresses further and the amount of uncracked wax fraction is reduced, meaning the flow rate of the bottom oil from the second fractionator 70 decreases, whereas as the reaction temperature of the wax fraction hydrocracking step is lowered, the amount of uncracked wax fraction increases, causing an increase in the flow rate of the bottom oil from the second fractionator 70. Accordingly, by raising the reaction temperature of the wax fraction hydrocracking step in those cases where the flow rate of the bottom oil from the second fractionator 70 is greater than normal, and lowering the reaction temperature of the wax fraction hydrocracking step in those cases where the flow rate of the bottom oil from the second fractionator 70 is less than normal, the wax fraction hydrocracking step can be maintained in a appropriate state. Provided the wax fraction hydrocracking step can be maintained in a appropriate state, the properties of the hydrocracked product from the wax fraction hydrocracking step can be stabilized, and the properties of the hydrocarbon oil supplied to the second fractionator 70 can also be stabilized, meaning the quality of the product obtained from the second fractionator 70 can be maintained at a favorable level.

In the wax fraction hydrocracking step, the reaction temperature is preferably set so that, as described above, the content of a specific hydrocarbon component in the hydrocracked product, namely that hydrocarbon component having a boiling point of not lower than 25° C. and not higher than 360° C., is preferably within a range from 20 to 90 mass %, more preferably from 30 to 80 mass % and still more preferably from 45 to 70 mass %, based on the total mass of the hydrocracked product having a boiling point of 25° C. or higher. An example is described below in which the operational target for the content of this specific hydrocarbon component is set to 67 mass %, and the bottom cut temperature of the second fractionator is set to 360° C.

The hydrocracking reaction temperature that yields a content of 67 mass % for the specific hydrocarbon component is designated as the standard reaction temperature. Under these conditions, the flow rate of the bottom oil from the second fractionator 70 is approximately 33% of the flow rate of the feed volume fed into the wax fraction hydrocracking reactor 50 (namely, the combination of the wax fraction from the FT synthesis reaction step and the recycled bottom oil). In other words, if the flow rate of the wax fraction from the FT synthesis reaction step is deemed 100, then the flow rate of the bottom oil is 50.

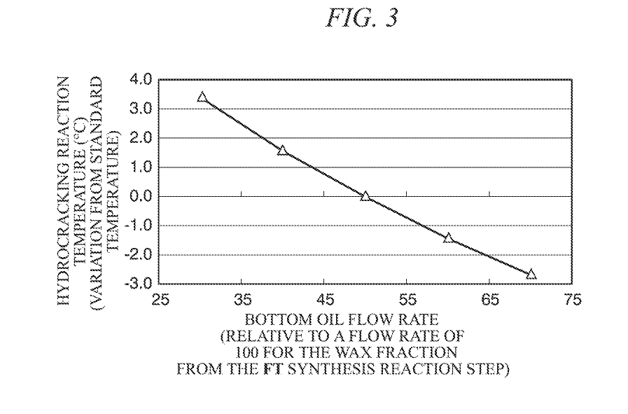

FIG. 3 is a graph illustrating the relationship between the ratio of the flow rate of the bottom oil relative to the flow rate of the wax fraction from the FT synthesis reaction step (the recycle ratio), and the reaction temperature (actual measured value) of the wax fraction hydrocracking step that gives such a bottom oil flow rate. In the graph, the horizontal axis represents the flow rate (on a volumetric basis) of the bottom oil, relative to a designated value of 100 for the flow rate of the wax fraction from the FT synthesis reaction step, which is supplied to the wax fraction hydrocracking step, either from the bottom of the first fractionator 40, or in some cases via the intermediate tank 62. The vertical axis represents the temperature variation in the wax fraction hydrocracking reaction temperature from the standard reaction temperature (±0° C.) at which the bottom oil flow rate (on the horizontal axis) is 50 (and the content of the above-mentioned specific hydrocarbon component is 67 mass %). In other words, FIG. 3 illustrates the relationship between the variation in the bottom oil flow rate from the standard value, and the variation in the reaction temperature from the standard temperature.

From this graph it is evident that when the flow rate of the bottom oil is high, the actual reaction temperature of the wax fraction hydrocracking step is lower than the standard reaction temperature. Accordingly, in this case, the process must be controlled so as to raise the reaction temperature of the wax fraction hydrocracking step. For example, if the flow rate of the bottom oil is 60, then reading from the graph indicates that the reaction temperature of the hydrocracking step has fallen 1.4° C. below the standard reaction temperature, and therefore an operation can be performed in the hydrocracking control step to raise the reaction temperature of the wax fraction hydrocracking step by 1.4° C. Further, the graph also reveals that when the flow rate of the bottom oil is low, the actual reaction temperature of the wax fraction hydrocracking step is higher than the standard reaction temperature. Accordingly, in this case, the process must be controlled so as to lower the reaction temperature of the hydrocracking step. For example, if the flow rate of the bottom oil is 40, then reading from the graph indicates that the reaction temperature of the wax fraction hydrocracking step has risen 1.6° C. above the standard temperature, and therefore an operation can be performed in the hydrocracking control step to lower the reaction temperature of the wax fraction hydrocracking step by 1.6° C.

By adjusting the reaction temperature in this manner, the wax fraction hydrocracking step can be controlled so as to achieve a content for the above-mentioned specific hydrocarbon component of 67 mass %, namely a bottom oil flow rate of 50.

In this manner, in the hydrocracking control step, the relationship between the flow rate of the bottom oil and the reaction temperature of the wax fraction hydrocracking step as illustrated in FIG. 3 is preferably ascertained in advance. Then, based on this relationship, the reaction temperature of the wax fraction hydrocracking step is preferably determined on the basis of the flow rate of the bottom oil, the reaction temperature then being adjusted to achieve the determined temperature. By controlling the process in this manner to return the flow rate of the bottom oil to a predetermined value, the wax fraction hydrocracking step can be rapidly returned to an appropriate operating state.

In this manner, if the process is controlled so that the bottom cut temperature in the second fractionator 70 is constant, then when the properties of the hydrocarbon oil supplied to the second fractionator 70 fluctuate, the flow rate of the bottom oil from the second fractionator 70 will also fluctuate. In order to better stabilize the wax fraction hydrocracking step against such fluctuations, the flow rate of the wax fraction from the FT synthesis reaction step is preferably adjusted in accordance with any fluctuations in the flow rate of the bottom oil, so that the combined flow rate of the wax fraction from the FT synthesis reaction step, which is supplied as new material to the wax fraction hydrocracking step, either from the first fractionator 40, or in some cases via the intermediate tank 62, and the resupplied bottom oil, namely the feed volume supplied to the wax fraction hydrocracking step, is maintained at a constant level. This ensures that the suppression effect achieved by performing control so that the bottom cut temperature in the fractionator is maintained at a constant value, which suppresses the vicious cycle that amplifies any fluctuation in the properties of the hydrocarbon oil supplied to the second fractionator 70, is more reliable.

In the hydrocracking control step, it is preferable that the relationship between the flow rate of the bottom oil and the reaction temperature of the wax fraction hydrocracking step is ascertained in advance, and the reaction temperature of the wax fraction hydrocracking step is then set to the temperature determined in accordance with the flow rate of the bottom oil on the basis of the ascertained relationship, and that, at the same time, the flow rate of the wax fraction is adjusted in accordance with the flow rate of the bottom oil, so that the combined flow rate (feed volume) of the wax fraction from the FT synthesis reaction step and the resupplied bottom oil is maintained at a constant level. By conducting the hydrocracking control step in this manner, if the properties of the hydrocarbon oil supplied to the second fractionator 70 fluctuate, then the vicious cycle that can cause the fluctuation to be amplified can be reliably suppressed, and the wax fraction hydrocracking step can be rapidly and reliably returned to a predetermined stable state.

As described above, by controlling the bottom cut temperature of the second fractionator 70 at a constant value in the fractional distillation step, and then, in the hydrocracking control step, controlling the wax fraction hydrocracking step in accordance with the fluctuations in the flow rate of the bottom oil caused by the controlling of the bottom cut temperature at a constant value, even if the properties of the hydrocarbon oil supplied to the second fractionator 70 fluctuate from the standard properties, the vicious cycle that causes the fluctuation to be amplified can be suppressed, enabling the properties of the hydrocarbon oil supplied to the second fractionator 70 to be stabilized and rapidly returned to the standard properties. As a result, the quality of the product obtained from the second fractionator 70 can be stably maintained.

In other words, by controlling the bottom cut temperature at a constant value in the fractional distillation step, the properties of the obtained bottom oil can be kept constant regardless of the properties of the hydrocarbon oil supplied to the second fractionator 70. By keeping the properties of the bottom oil constant in this manner, the properties of the hydrocracked product obtained in the wax fraction hydrocracking step that is supplied with the bottom oil also settle to a constant level. Further, by controlling the fractional distillation step in this manner, the flow rate of the bottom oil fluctuates in accordance with the properties of the hydrocarbon oil supplied to the second fractionator 70, and therefore in addition to controlling the fractional distillation step in the manner described above, the reaction conditions for the wax fraction hydrocracking step are controlled using the flow rate of the bottom oil as an indicator. This enables the degree of progression of the hydrocracking in the wax fraction hydrocracking step to be appropriately controlled, meaning the properties of the hydrocracked product obtained in the wax fraction hydrocracking step can be maintained at a constant level.

By controlling the bottom cut temperature of the second fractionator 70 at a constant temperature, as well as controlling the wax fraction hydrocracking step on the basis of the flow rate of the bottom oil, the wax fraction hydrocracking step can be controlled appropriately against both fluctuations in the raw material supplied to the wax fraction hydrocracking step, and fluctuations in the reaction within the wax fraction hydrocracking step, meaning the properties of the product can be stably maintained.

While preferred embodiments of the present invention have been described and illustrated above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

In the above embodiments, a liquid fuel synthesizing system 1 used within a plant for converting a natural gas as a hydrocarbon feed stock to a base stocks for liquid fuels was described, but the present invention is not only for application to those cases where a natural gas is used as a feedstock, and can also be applied to cases that use hydrocarbons other than natural gas, such as asphalt and residual oils, as a feedstock. In other words, the present invention can be applied to any system that synthesizes liquid hydrocarbons by an FT synthesis reaction that involves bringing a feedstock gas containing at least carbon monoxide gas and hydrogen gas into contact with a catalyst slurry, and from the obtained liquid hydrocarbons, produces hydrocarbon oils to be used for liquid fuel base stocks or the like.

In the process for producing a hydrocarbon oil of the present invention, a “hydrocarbon oil” refers to a hydrocarbon oil containing a hydrocracked product of wax fraction produced by the hydrocracking process of the invention, a naphtha fraction or middle distillate obtained by fractional distillation of the hydrocracked product, a kerosene fraction and gas oil fraction obtained by fractional distillation of the middle distillate, or a mixture thereof.

### INDUSTRIAL APPLICABILITY

The present invention relates to a hydrocracking process for a wax fraction that includes a wax fraction hydrocracking step of hydrocracking a wax fraction contained within liquid hydrocarbons synthesized by a Fischer-Tropsch synthesis reaction, thereby obtaining a hydrocracked product, a fractional distillation step of supplying the hydrocracked product to a fractionator in which a bottom cut temperature is set to a constant value, and obtaining at least a middle distillate and a bottom oil from the fractionator, a recycling step of resupplying all of the bottom oil to the wax fraction hydrocracking step, and a hydrocracking control step of controlling the wax fraction hydrocracking step using a flow rate of the bottom oil as an indicator, and also relates to a process for producing a hydrocarbon oil using said hydrocracking process.

According to the present invention, the stability of the product obtained from the fractionator can be stably maintained.

### DESCRIPTION OF THE REFERENCE SIGNS

70: Second fractionator
50: Wax fraction hydrocracking reactor

1. A hydrocracking process for a wax fraction, comprising:
a wax fraction hydrocracking step of hydrocracking a wax fraction contained within liquid hydrocarbons synthesized by a Fischer-Tropsch synthesis reaction, thereby obtaining a hydrocracked product,
a fractional distillation step of supplying said hydrocracked product to a fractionator in which a bottom cut temperature is set to a constant value, and obtaining at least a middle distillate and a bottom oil from said fractionator,
a recycling step of resupplying all of said bottom oil to said wax fraction hydrocracking step, and
a hydrocracking control step of controlling said wax fraction hydrocracking step using a flow rate of said bottom oil as an indicator.

2. The hydrocracking process for a wax fraction according to claim 1, wherein
said hydrocracking control step is a step in which a relationship between a flow rate of said bottom oil and a reaction temperature of said wax fraction hydrocracking step is ascertained in advance, and said reaction temperature is set in accordance with said flow rate of the bottom oil based on said relationship.

3. The hydrocracking process for a wax fraction according to claim 1 or claim 2, wherein
said hydrocracking control step is a step in which said flow rate of the wax fraction is adjusted in accordance with a flow rate of said bottom oil, so that a combined flow rate of said wax fraction that is supplied to said wax fraction hydrocracking step and said bottom oil that is resupplied to said wax fraction hydrocracking step remains constant.

4. A process for producing a hydrocarbon oil, comprising:
a liquid hydrocarbon synthesis step of synthesizing liquid hydrocarbons from a feedstock gas comprising carbon monoxide gas and hydrogen gas by a Fischer-Tropsch synthesis reaction,
a wax fraction hydrocracking step of hydrocracking a wax fraction contained within said liquid hydrocarbons synthesized in said liquid hydrocarbon synthesis step, thereby obtaining a hydrocracked product,
a fractional distillation step of supplying said hydrocracked product to a fractionator in which a bottom cut temperature is set to a constant value, and obtaining at least a middle distillate and a bottom oil from said fractionator,
a recycling step of resupplying all of said bottom oil to said wax fraction hydrocracking step, and
a hydrocracking control step of controlling said wax fraction hydrocracking step using a flow rate of said bottom oil as an indicator.

5. The process for producing a hydrocarbon oil according to claim 4, wherein
said hydrocracking control step is a step in which a relationship between a flow rate of said bottom oil and a reaction temperature of said wax fraction hydrocracking step is ascertained in advance, and said reaction temperature is set in accordance with said flow rate of the bottom oil based on said relationship.

6. The process for producing a hydrocarbon oil according to claim 4 or claim 5, wherein
said hydrocracking control step is a step in which a flow rate of said wax fraction is adjusted in accordance with said flow rate of said bottom oil, so that a combined flow rate of said wax fraction that is supplied to said wax fraction hydrocracking step and said bottom oil that is resupplied to said wax fraction hydrocracking step remains constant.
